(19) United States
(12) Patent Application Publication
(10) Pub. No.: US 2018/0353530 A1
(43) Pub. Date: Dec. 13, 2018

(54) Combination Therapeutic Agent For The Treatment Of Macular Degeneration

(71) Applicant: Wolfgang PRIES, Worishofen (DE)

(72) Inventor: Wolfgang PRIES, Worishofen (DE)

(21) Appl. No.: 16/005,854

(22) Filed: Jun. 12, 2018

(30) Foreign Application Priority Data
Jun. 13, 2017 (DE) 102017005546.7

(51) Int. Cl. A61K 31/7084; A61P 27/02; A61K 9/00; A61K 9/48

(52) U.S. Cl. CPC A61K 31/7084; A61P 27/02; A61K 33/30; A61K 9/48; A61K 2300/00; A61K 9/0019

(57) ABSTRACT

A combination therapeutic agent is described which comprises reduced nicotinamide adenine dinucleotide (NADH) or a salt thereof and at least one antioxidative additive agent for use in the treatment of macular degeneration.

### BACKGROUND OF THE INVENTION

### 1. Field of the Invention

The invention is directed to a combination therapeutic agent comprising reduced nicotinamide adenine dinucleotide (NADH) or a salt thereof and at least one antioxidative additive agent for use in the treatment of macular degeneration.

### 2. Description of the Related Art

The macula designates a small area located in the center of the retina of the eye. In adult humans, the diameter of the macula is approximately 3 mm to 5 mm. In the center of the macula lies the fovea, the location of highest visual acuity with the highest density of color-sensitive sensory cells, also called cones.

Macular degeneration designates a group of macular retinal disorders. A loss in function of the various cells located in the macula results in a decline of central visual acuity. Often, both eyes are affected. However, the disease can progress differently in the two eyes. It is also possible for only one eye to be affected.

The most common form of macular degeneration is senile or age-related macular degeneration (AMD). This refers to a painless, progressive change in vision within the central field of vision. Initially, patients suffer from a blurry, gradually enlarging dark spot in the center of the visual field. Further, there is a decrease in contrast sensitivity, color vision and light-to-dark adaptation. Sensitivity to glare is heightened, and contours appear blurry and distorted (metamorphopsia).

Age-related macular degeneration is the leading cause of blindness in persons over fifty years of age and accounts for approximately 32% of new blindness, although the peripheral field of vision is retained. Due to the changing age composition of the population with a growing proportion of elderly people, the number of new cases of macular degeneration has substantially increased. It is estimated that 25 to 30 million people worldwide are afflicted with macular degeneration.

Approximately 500,000 new cases of macular degeneration are reported every year. In Germany, an estimated 4 million people suffer from macular degeneration. The loss of vision which can progress to blindness, severely limits the autonomy of a patient so that social problems can be foreseen with respect to costs of insurance and care owing to age-related macular degeneration.

There are two distinct forms of age-related macular degeneration: the more common, slowly progressing dry form and the more aggressive wet AMD. Further, dry AMD has an early stage and a late stage. The disease is promoted by the formation of drusen, i.e., small yellow deposits under the retina and changes to the pigment epithelium on the ocular fundus. Drusen are formed, e.g., as a result of oxidative stress and poor microcirculation, followed by accumulations of extracellular material such as immune complexes, proteins or lipids under the retina. Small, hard drusen occur in over 98% of the total population, even in healthy individuals, and can break down again. Soft drusen, in contrast, are usually not observed until old age. Preexisting damage to the retinal pigment epithelium contributes to the occurrence of soft drusen. Drusen can further promote irreversible damage to the pigment epithelium. Degeneration in the form of functional and/or morphological changes and atrophy of the pigment epithelium lead to a degeneration of the cells located above the drusen. The occurrence of soft drusen usually promotes the onset of AMD. Drusen do not impair visual acuity but cause impairment of color sensitivity and contrast sensitivity. In the early stage, vision is only slightly impaired and the changes only progress gradually. In the late stage, sensory cells and their feeding cells are destroyed. Patients with advanced-stage dry AMD are also at higher risk for developing the wet form of AMD.

The more aggressive, wet AMD is linked to a deterioration of visual acuity. In a small number of patients, drusen were also determined. Wet AMD is caused by the formation of new, abnormal blood vessels that grow as small vascular buds under the retina. Since these vessels are leaky, vascular fluid escapes into the retina, where it leads to swelling of the macula resulting in macular edema and hemorrhaging in the unstable vascular system. Scarring causes a permanent loss of visual acuity.

The therapy is governed by the form of macular degeneration. A photodynamic therapy (PDT) is used for wet AMD, as are vascular growth inhibitors (VEGF inhibitors) that are injected directed into the eyeball where they inhibit angiogenesis. Further, vessels can be sealed by percutaneous laser irradiation, and hemangiomas can be eliminated through retinal surgery. These treatments are complicated, entail high treatment costs and must be repeated at individualized intervals. In conventional medicine, there has not so far been any standardized therapy for dry AMD. The loss of vision cannot be restored. In order to prevent the macular degeneration from progressing, a vitamin-rich diet is recommended for patients but is not sufficiently effective. For this reason, an improved and/or more efficient therapy is required for the treatment and prophylaxis of macular degeneration.

### SUMMARY OF THE INVENTION

The present invention is directed to a combination therapeutic agent comprising reduced nicotinamide adenine dinucleotide (NADH) or a salt thereof and at least one antioxidative additive agent for use in the treatment of macular degeneration.

Further, the combination therapeutic agent for use according to the invention is administered over an infusion interval on consecutive days, where NADH is administered in oral form followed immediately by the at least one antioxidative additive agent in the form of an intravenous infusion for a duration of approximately 1 to 2 hours followed immediately by NAND in oral form.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### DETAILED DESCRIPTION OF THE PRESENTLY PREFERRED EMBODIMENTS

In a first aspect, the invention is directed to a combination therapeutic agent comprising reduced nicotinamide adenine dinucleotide (NADH) or a salt thereof and at least one antioxidative additive agent for use in the treatment of macular degeneration.

The term “combination therapeutic agent” as used herein means an agent having two or more antioxidative substances. The advantage of the combination therapeutic agent consists in an improved primary antioxidative action, an expanded spectrum of activity through organic and inorganic antioxidants with very strong intracellular and extracellular antioxidant activity, and an improvement in the pharmacokinetic parameters of an antioxidative therapy. A particular advantage consists in that the utilized antioxidative substances achieve a synergistic effect. The combination therapeutic agent achieves a higher efficiency than when individual antioxidative substances are used. Therapy with the combination therapeutic agent is comparatively inexpensive and is well tolerated. The combination therapeutic agent is composed exclusively of substances that occur naturally in the human body. The antioxidative substances can be present together in a preparation or in different preparations of the same and/or different dosage forms. “Preparation” means a product composed of at least one antioxidative additive agent and excipients. The liquid dosage form includes, for example, a solution, a suspension or an emulsion. The solid dosage form includes, for example, a capsule or a tablet. It is advantageous in this connection that the active ingredient is in optimal dosage form in accordance with its physicochemical properties. This ensures a particularly good storability and shelf life prior to use and prevents undesirable pharmacological, physiological and/or physical-chemical interactions with each other or with the preparation container. This prevents disadvantageous, degrading and undesirable effects on the antioxidative substance. The term “reduced nicotinamide adenine dinucleotide” or “NADH” as used herein refers to a coenzyme of hydrogen donor enzymes. It is a powerful antioxidant produced by the body and transfers a proton and two electrons. NADH protects cellular and extracellular substances and structures from the harmful effect of oxygen radicals in the body. It improves microcirculation. In addition, NADH contributes to cellular energy production. Depending on dosage, NADH also stimulates the biosynthesis of Interleukin-6 which has a neuroprotective effect against a variety of cell damage, in particular of nerve cells.

The term “antioxidative additive agent” as used herein means a substance that exhibits antioxidant activity and is used in addition to the NADH. An antioxidative substance even in a low concentration prevents or delays the oxidation of other substances and structures in the cell. The antioxidative additive agent has a lower redox potential than the substance or structure to be protected in the cell and is therefore oxidized before the substance or structure to be protected in the cell. The antioxidative additive agent works synergistically together with NADH. The antioxidative additive agent assists NADH in the protective effect on cellular and extracellular substances and structures against the damaging effect of oxygen radicals in the body. Oxidative stress due to free oxygen radicals promotes macular degeneration. If oxidative stress is reduced, it is possible to slow the progress of macular degeneration. The different combinations of antioxidative additive agents show a lesser antioxidative effect compared with antioxidative additive agents combined with NADH. Surprisingly, every combination of NADH and antioxidative additive agents shows not only a prophylactic effect against oxidative stress but also a therapeutic effect on macular degeneration. The symptoms of macular degeneration measured in visual acuity, for example, improved by up to 20% and in some patients by up to 50% depending on treatment duration when a combination of NADH and antioxidative additive agents was used. No side effects were observed. Consequently, therapy with the combination therapeutic agent according to the invention is advantageous in the treatment of AMD. Further, the combination therapeutic agent can reduce the risk of a progression from dry AMD to wet AMD. The combination therapeutic agent can also reverse wet AMD to dry AMD.

In a preferred embodiment form, the combination therapeutic agent for use according to the invention is administered over an infusion interval on consecutive days, wherein NADH is administered in oral form followed immediately by the at least one antioxidative additive agent in the form of an intravenous infusion for a duration of approximately 1 to 2 hours followed immediately by NADH in oral form. NADH is preferably used in capsule form at the start of the regimen and at end of the regimen.

The term “infusion interval” as used herein means a particular duration determined on an individual basis in which the above-mentioned regimen is applied. The infusion interval can be repeated at intervals determined on an individual basis over the same duration or over a shorter duration or over a longer duration. The infusion interval is preferably repeated every 8 weeks. More preferably, the infusion interval is repeated every 4 weeks, more preferably every 3 weeks. Depending on the infusion rate, the infusion duration over which the infusion solution is administered completely to the patient is between 1 and 2 hours, preferably 1 hour. The patient's blood pressure is monitored repeatedly over the infusion duration. There is advantageously a one-hour period of rest after infusion cessation. A bioavailability of 100% is achieved for the respective utilized antioxidative substances through intravenous administration. Intravenous administration allows higher dosages of the antioxidative substances than, e.g., peroral administration.

In a preferred embodiment form, the combination therapeutic agent is administered from 3 to 8 consecutive days, preferably 5 consecutive days. The infusion interval is governed by the stage and form of macular degeneration. In the advanced stage of dry AMD, a longer infusion interval is advantageous. It is advantageous to determine visual acuity at the end of every infusion interval. This allows a therapy specifically adapted to the patient.

In a preferred embodiment form, NADH or a salt thereof is in the form of a capsule, preferably a gastro-resistant capsule. NADH is an acid-unstable solid. NADH is preferably used as a gastro-resistant capsule to impede the disintegration of NADH in gastric acid. It is advantageous that NADH is absorbed into the body intact and, therefore, in a greater amount.

Alternatively, NADH can also be added to the infusion solution shortly before administration. Further, NADH can also be used in the form of a second infusion solution administered separately or added continuously to the first infusion solution with the antioxidative additive agents.

In a preferred embodiment form, the content of NADH or a salt thereof corresponds to 10 to 1000 mg of NADH, preferably 10 to 250 mg, more preferably 10 to 80 mg, most preferably 20 mg per dosage unit. The term “dosage unit” as used herein refers to a dosage form for one-time use, e.g., 1 capsule or 1 infusion solution. The amount of NADH to be administered is governed on an individual basis by the form and stage of macular degeneration and by the existing antioxidant status in the body of the patient, i.e., the antioxidant capacity as it is called. The antioxidant capacity is the sum of all endogenous and exogenous defense mechanisms against oxidative stress in the organism. Preferably, a total amount of approximately 20 mg to 160 mg of NADH, more preferably 40 mg to 80 mg of NADH is administered daily.

In a preferred embodiment form, the at least one antioxidative additive agent is ascorbic acid, glutathione, α-lipoic acid, selenium, zinc, folic acid, vitamin B12, magnesium, potassium and/or L-arginine or a derivative or salt thereof. NADH is combined with at least one antioxidative additive agent. This has the advantage that the combination with NADH exhibits a synergistic effect far exceeding the effects of the individual substances. By administering a combination of different antioxidative additive agents with NADH, surprisingly significant improvements in the visual acuity of the patients were achieved. The combination therapeutic agent can also contain a plurality, in particular a large number of, antioxidative additive agents in addition to NADH. The individual antioxidative additive agents are preferably added in determined amounts and ratios. More preferably, the antioxidative additive agents can be added in ratios which are to be determined on an individual basis. This has the advantage that the dosages of the individual substances in the combination therapeutic agent can be tailored to the form and stage of macular degeneration and to the antioxidant status of the patient.

In a preferred embodiment form, the antioxidative additive agent is ascorbic acid or a salt thereof and the content corresponds to 100 to 2000 mg, preferably 600 to 1000 mg, more preferably 800 to 1000 mg, most preferably 1000 mg of ascorbic acid per dosage unit. The amount of ascorbic acid to be administered on an individual basis is governed by the above-described parameters. Preferably, a total amount of approximately 1000 mg of ascorbic acid is administered per day of treatment. Ascorbic acid has a high redox potential. The redox potential is a measurement of the reduction-oxidation capacity of a substance. The more negative the redox potential, the greater the reduction force. Ascorbic acid acts as a cofactor of numerous enzyme systems and is an important component of immune defense. Ascorbic acid preferably builds up in the eye lens. Accordingly, ascorbic acid has a very good antioxidative action not only in the blood but also in the eye.

In a preferred embodiment form, the antioxidative additive agent is glutathione or a salt thereof, and the content corresponds to 100 to 1500 mg, preferably 100 to 1000 mg, more preferably 500 to 800 mg, most preferably 600 mg of glutathione per dosage unit. The amount of glutathione to be administered on an individual basis is governed by the above-described parameters. Preferably, a total amount of approximately 600 mg of glutathione is administered per day of treatment. Glutathione (GSH) is a tripeptide comprising the three amino acids glutamic acid, cysteine, and glycine. In its antioxidative activity, glutathione is oxidized and changes from its monomeric form GSH to a dimer GSSG.

In a preferred embodiment form, the antioxidative additive agent is α-lipoic acid or a salt thereof, and the content corresponds to 100 to 1500 mg, preferably 100 to 1000 mg, more preferably 500 to 800 mg, most preferably 600 mg of α-lipoic acid per dosage unit. The amount of α-lipoic acid to be administered on an individual basis is governed by the above-described parameters. Preferably, a total amount of approximately 600 mg of α-lipoic acid is administered per day of treatment. The reduced form of α-lipoic acid, dihydrolipoic acid, as dithiol, has strong antioxidative properties.

In a preferred embodiment form, the antioxidative additive agent is a selenium salt and the content corresponds to 50 μg to 500 μg, preferably 100 μg to 300 μg, more preferably 200 μg to 300 μg, most preferably 300 μg of selenium per dosage unit. The amount of selenium to be administered on an individual basis is governed by the above-described parameters. Preferably, a total amount of approximately 300 μg of selenium is administered per day of treatment. Selenium in the form of selenocysteine is an amino acid which occurs, for example, in the active site of the enzyme glutathione peroxidase. Glutathione peroxidase is a component of the cellular defense against oxidative stress. Further, selenium is an important radical trap due to high reactivity with oxygen.

In a preferred embodiment form, the antioxidative additive agent is a zinc salt and the content corresponds to 1 mg to 100 mg, preferably 5 mg to 50 mg, more preferably 10 mg to 25 mg of zinc per dosage unit. The amount of zinc to be administered on an individual basis is governed by the above-described parameters. Preferably, a total amount of approximately 10 mg of zinc is administered per day of treatment. Zinc plays an important role as a structural, catalytic or regulatory component of enzymes (e.g., dehydrogenases), of sequestrants and in cellular and humoral immune response. The highest concentrations of zinc are found in the fundus. Accordingly, zinc is particularly well suited to have an antioxidative effect in the eye.

In a preferred embodiment form, the antioxidative additive agent is folic acid or a derivative or a salt thereof and the content corresponds to 100 μg to 10 mg, preferably 1 mg to 10 mg, more preferably 3 mg to 6 mg, and most preferably 5 mg of folic acid per dosage unit. The amount of folic acid to be administered on an individual basis is governed by the above-described parameters. Preferably, a total amount of approximately 5 mg of folic acid is administered per day of treatment. Folic acid plays a part in growth processes and in cell division.

In a preferred embodiment form, the antioxidative additive agent is vitamin B12 or a derivative thereof and the content corresponds to 100 to 1500 mg, preferably 100 to 1000 mg, more preferably 500 to 1000 mg, most preferably 1000 mg of vitamin B12 per dosage unit. The amount of vitamin B12 to be administered on an individual basis is governed by the above-described parameters. Preferably, a total amount of approximately 1000 mg of vitamin B12 is administered per day of treatment. Vitamin B12 plays an important role in various metabolic processes. For example, vitamin B12 aids in the breakdown of certain fatty acids. Further, vitamin B12 aids hematopoiesis by converting the folic acid stored in the organism into its active form.

In a preferred embodiment form, the antioxidative additive agent is a magnesium salt and the content corresponds to 100 to 1000 mg, preferably 100 mg to 500 mg, more preferably 400 mg to 500 mg of magnesium per dosage unit. The amount of magnesium to be administered on an individual basis is governed by the above-described parameters. Preferably, a total amount of approximately 500 mg of magnesium is administered per day of treatment. Magnesium is needed by more than 300 enzymes as enzyme component or coenzyme and in this way exerts influence on cell regeneration, oxygen usage and energy production. Moreover, magnesium stabilizes biological membranes.

In a preferred embodiment form, the antioxidative additive agent is a potassium salt and the content corresponds to 100 mg to 1000 mg, preferably 500 mg to 1000 mg, more preferably 600 to 800 mg, most preferably 780 mg of potassium per dosage unit. The amount of potassium to be administered on an individual basis is governed by the above-described parameters. Preferably, a total amount of approximately 780 mg of potassium is administered per day of treatment. Potassium participates in a large number of physiological processes in the body, for example, regulation of cell growth, influencing protective endothelial vascular functions, influencing the release of hormones, carbohydrate utilization and protein synthesis.

In a preferred embodiment form, the antioxidative additive agent is L-arginine or a salt thereof and the content corresponds to 100 mg to 2000 mg, preferably 300 mg to 1000 mg, more preferably 300 to 600 mg, most preferably 450 mg of L-arginine per dosage unit. The amount of L-arginine to be administered on an individual basis is governed by the above-described parameters. Preferably, a total amount of approximately 900 mg of L-arginine is administered per day of treatment. It is only from arginine that the body can form the vessel-activating molecule nitrogen (NO) which controls vasodilation and, therefore, blood flow and blood pressure. Accordingly, arginine is an essential amino acid.

In a preferred embodiment form, the antioxidative additive agents are zinc and/or selenium administered at staggered times. The content of selenium salt corresponds to 50 μg to 500 μg, preferably 100 μg to 300 μg, more preferably 200 μg to 300 μg, most preferably 300 μg of selenium per dosage unit and/or the content of the zinc salt corresponds to 1 mg to 100 mg, preferably 5 mg to 50 mg, more preferably 10 mg to 25 mg of zinc per dosage unit. The term “staggered times” as used herein means that selenium and zinc are administered at intervals of 3 to 4 hours.

In a preferred embodiment form, the antioxidative additive agents zinc and/or selenium are administered orally. The patient takes zinc and/or selenium in oral form, e.g., in capsule or tablet form, between the infusion intervals. This has the advantage that the patient can also maintain or further build up his/her antioxidant status between the infusion intervals without direct supervision of the physician. This improves the successful outcome of the therapy.

In a preferred embodiment form, the antioxidative additive agents zinc and/or selenium are/is preferably administered daily and preferably for 8 weeks, more preferably for 4 weeks, more preferably for 3 weeks after an infusion interval. The frequency and duration of administration is governed by the form and stage of macular degeneration and by the antioxidant status of the patient.

In a preferred embodiment form, the antioxidative additive agents are ascorbic acid and glutathione. The combination of ascorbic acid and glutathione with NADH has a synergistic effect compared with the respective individual substances. The amount of ascorbic acid and glutathione to be administered on an individual basis is governed by the parameters already described. A total amount of approximately 1000 mg of ascorbic acid and a total amount of approximately 600 mg of glutathione is preferably administered each day of treatment. The administration of antioxidative additive agents with various antioxidative mechanisms potentiates the efficacy of the combination therapeutic agent.

In a preferred embodiment form, the antioxidative additive agents are ascorbic acid and magnesium. The combination of ascorbic acid and magnesium has a synergistic effect compared with the respective individual substances. It is advantageous when ascorbic acid and magnesium are administered simultaneously. An optimal, synergistic effect of ascorbic acid and magnesium is achieved in this way. The amount of ascorbic acid and magnesium to be administered on an individual basis is governed by the parameters already described. Preferably, a total amount of approximately 1000 mg of ascorbic acid and a total amount of approximately 500 mg of magnesium is preferably administered each day of treatment. Magnesium and ascorbic acid together form a complex which improves the absorption of the resulting magnesium ascorbates into the cells.

In a preferred embodiment form, the antioxidative additive agents ascorbic acid and magnesium are administered simultaneously. The content of ascorbic acid corresponds to 100 to 2000 mg, preferably 600 to 1000 mg, more preferably 800 to 1000 mg, most preferably 1000 mg of ascorbic acid per dosage unit. The content of magnesium corresponds to 100 mg to 1000 mg, preferably 100 mg to 500 mg, more preferably 400 mg to 500 mg of magnesium per dosage unit. Simultaneous administration enhances the complexation of magnesium ascorbate and, therefore, the proportion of magnesium and ascorbic acid absorbed in the cells.

In a preferred embodiment form, the antioxidative additive agents are ascorbic acid, glutathione, α-lipoic acid and vitamin B12. The combination of these additive agents has a synergistic effect in comparison with the respective individual substances. The different antioxidative mechanisms of the antioxidative additive agents potentiates the efficacy of the combination therapeutic agent.

NADH and the antioxidative additive agents are dissolved and diluted in a basic solution. This basic solution is advantageously a sterile normal saline solution (0.9%) in an amount of from 50 ml to 500 ml, preferably from 50 ml to 250 ml. The normal saline solution is preferably used for patients with renal and/or cardiac insufficiency and for patients with cardiac decompensation. Further, the basic solution is advantageously a sterile colloidal solution of hydroxyethyl starch (HES). HES is preferably present in a sterile electrolyte solution. The content of HES is preferably 6%. The electrolytes include sodium, potassium, calcium, magnesium, chloride, acetate and L-malate in physiological concentrations. The amount of HES solution used is preferably 250 ml to 500 ml.

### EXAMPLES

### Example 1

Production of the Infusion Solution and Use Thereof Together with NADH as Combination Therapeutic Agent

The infusion solution is produced from commercially available liquid and sterile injection solutions of the individual antioxidative additive agents.

### Example 1a

The contents of an ampoule of ascorbic acid (1000 mg in 5 ml) and glutathione (600 mg in 4 ml) are added to 250 ml of a sterile HES solution.

### Example 1b

The contents of an ampoule of ascorbic acid (1000 mg in 5 ml) and magnesium sulfate (corresponding to 493 mg of magnesium in 10 ml) are added to 250 ml of a sterile HES solution.

### Example 1c

The contents of an ampoule of ascorbic acid (1000 mg in 5 ml), glutathione (600 mg in 4 ml), α-lipoic acid (600 mg in 24 ml) and vitamin B12 (1000 μg in 2 ml) are added to 250 ml of a sterile HES solution.

### Example 1d

The contents of an ampoule of ascorbic acid (1000 mg in 5 ml), glutathione (600 mg in 4 ml), α-lipoic acid (600 mg in 24 ml), sodium selenite (corresponding to 300 μg of selenium in 1 ml), zinc gluconate (corresponding to 10 mg of zinc in 2 ml), folic acid (5 mg in 2 ml), vitamin B12 (1000 μg in 2 ml), magnesium sulfate (corresponding to 493 mg of magnesium in 10 ml) and potassium chloride (corresponding to 780 mg of potassium in 20 ml) are added to 250 ml of a sterile HES solution.

TABLE 1
Composition of the infusion solutions of Examples 1a to 1d.

| | Example | | | |
|---|---|---|---|---|
| Component | 1a | Example 1b | Example 1c | Example 1d |
| Ascorbic acid | 1000 mg | 1000 mg | 1000 mg | 1000 mg |
| Glutathione | 600 mg | | 600 mg | 600 mg |
| α-Lipoic acid | | | 600 mg | 600 mg |
| Sodium selenite | | | | 300 μg |
| Zinc gluconate | | | | 10 mg |
| Folic acid | | | | 5 mg |
| Vitamin B12 | | | 1000 μg | 1000 μg |
| Magnesium | | 493 mg | | 493 mg |
| sulfate | | | | 780 mg |
| Potassium | 250 ml | 250 ml | 250 ml | 250 ml |
| chloride | | | | |
| HES solution | | | | |

Use

Infusion solutions of this composition are administered to the patient for 5 consecutive days for an infusion duration of 1 hour together with NADH and L-arginine, both in capsule form (one at the start of infusion and one at the end of infusion). The patient rests for 1 hour after the infusion.

### Example 2

Selection of the Basic Solution

Patients with AMD are usually elderly with multiple morbidities. Therefore, a clinical examination, determination of various laboratory parameters and an EKG are carried out before using the combination therapeutic agent. Renal insufficiency and/or cardiac insufficiency will be detected in this way, which determines the selection of the basic solution. In case of renal and/or cardiac insufficiency, the use of a volume-expanding HES solution is not considered; rather, a normal saline solution which has no volume-expanding effect is used as basic solution so as not to cause a rise in blood pressure and stress the cardiovascular system.

If the EKG shows a steady heart rate of below 55 beats per minute, a smaller dose of magnesium, about 500 mg, must be added so as not to further lower the heart rate.

### Example 3

Ophthalmological Studies and Findings

The patient collective of 63 patients aged 35 to 87 years is divided into three groups. Different regimens are used based on the group classification. The stage is determined based on examinations such as visual acuity (ETDRS letter charts, Landolt rings), distortion (Amsler grid), contrast vision and changes to the retina (funduscopy, fluorescence angiogram, optical coherence tomography). The groups are made up of patients with early-stage to late-stage dry AMD and wet AMD. The minimum visual acuity in all patients was 0.4 (ETDRS equivalent). This gives a picture of a clinically relevant basic population. A prospective, placebo-controlled and double-blind study is carried out.

Group 1 was given the respective combination therapeutic agent for 7 days, and group 2 was given the combination therapeutic agent for 7 days, plus the additional administration of zinc and selenium for 3 weeks after the infusion interval. Group 3 was given the basic solution without further additions as placebo for 7 days. The medical examination was always performed on both eyes prior to the start of therapy, shortly before the start of the further infusion interval and after the infusion interval. The study was carried out for a period of 18 months. The infusion interval was repeated in a 4-week cycle within this time period.

TABLE 2
Improvement in the visual acuity [%] of the test subjects after a
determined number of infusion intervals using combination
therapeutic agent 1b.

| Combination therapeutic | Number | Improvement in |
|---|---|---|
| agent 1b | of test subjects | visual acuity |
| 2 infusion intervals without | 12 | 10-20% |
| additional administration of | 22 | 0% |
| zinc and selenium | 4 | Worsening of up to 3% |
| 2 infusion intervals with | 5 | up to 28% |
| additional administration of | | |
| zinc and selenium | | |
| 18 infusion intervals with | 20 | up to 50% |
| additional administration of | | |
| zinc and selenium | | |

As a result of the infusion treatment with the combination therapeutic agent having the composition indicated in Example 1 b, the visual acuity of 12 test subjects improved by from 10 to 20% already after two infusion intervals. The number and density of drusen were also reduced. An improvement in visual acuity of up to 28% was determined in 5 patients with AMD to whom zinc and selenium were administered orally for a duration of 3 weeks following the infusion interval. Stabilization of visual acuity was seen in 22 patients. The visual acuity of 4 patients decreased by up to 4% over the course of treatment. In 20 patients the visual acuity improved by up to 50% after 18 infusion intervals and oral administration of zinc and selenium between the infusion intervals.

This shows that combination therapeutic agent 1 b slows the progress of macular degeneration and also brings about a healing process of macular degeneration. Consequently, combination therapeutic agent 1b reduces the risk of a progression from dry AMD to wet AMD. Also, as a result of the combination therapeutic agent, wet AMD may revert back to dry AMD. Other disease-related impairments and, therefore, the subjective wellbeing of the patient were likewise improved.

### Example 4

Determination of the Total Antioxidant Capacity of the Blood

The total antioxidant capacity is measured before the start of therapy and also at regular intervals shortly before the start of the further infusion interval and again at the end of the infusion interval.

Intracellular antioxidant capacity is chiefly ascribed to enzymatic reactions, whereas in the extracellular arena it is chiefly antioxidants supplied to the body which perform this function. Plasma antioxidant capacity may be considered as representative of the natural bodily equilibrium of oxidizing and antioxidative compounds. The laboratory study is conducted using the TAS test (Total Antioxidant Status test, commercially available from Randox Laboratories). The test uses ABTS, 2,2′-Azino-bis(3-ethylbenzothiazoline-6-sulfonic acid), and hydrogen peroxide to generate ABTS radical cations in the presence of metmyoglobin (HX-FE(III)) as peroxidase. Antioxidants in the plasma sample reduce the formation of radical cations with blue-green color. The extent of coloration is determined by absorption spectroscopy at a wavelength of 600 nm.

The antioxidant capacity of the blood averages in the majority of patients is between 1.30 and 1.77 mmol/l. A correlation between the existing antioxidant capacity and response to the combination therapeutic agent measurable by an improvement in visual acuity was not observed. Therefore, the outstanding success of the antioxidative combination therapeutic agent does not depend on the antioxidant status of the patient. However, the antioxidant capacity is taken into account for the dosage of the combination therapeutic agent so as to avoid an excessive dose of the antioxidative substances.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method for treating a patient suffering from macular degeneration, comprising:
administering to the patient a therapeutically effective amount of a combination of therapeutic agents comprising:
nicotinamide adenine dinucleotide hydride (NADH) or a salt thereof; and
at least one antioxidative additive agent.

2. The method according to claim 1, wherein the combination of therapeutic agents is administered over an infusion interval on consecutive days and comprises:
administering the NADH in oral form;
administering the at least one antioxidative additive agent in the form of an intravenous infusion for a duration of approximately 1 to 2 hours immediately following the administering the NADH in oral form; and
administering the NADH in oral form immediately following the administering of the at least one antioxidative additive agent.

3. The method according to claim 2, wherein the administering is carried out for at least one of:
3 to 8 consecutive days and
5 consecutive days.

4. The method according to claim 1, wherein the NADH or the salt thereof is one of:
a capsule and
a gastro-resistant capsule.

5. The method according to claim 1, wherein the NADH or the salt thereof is administered in a dose of at least one of:
10 to 1000 mg of NADH per dosage unit,
10 to 250 mg of NADH per dosage unit,
10 to 80 mg of NADH per dosage unit, and
20 mg of NADH per dosage unit.

6. The method according to claim 1, wherein the at least one antioxidative additive agent is one or more of ascorbic acid, glutathione, α-lipoic acid, selenium, zinc, folic acid, vitamin B12, magnesium, potassium and L-arginine, or a derivative or salt thereof.

7. The method according to claim 1, wherein the at least one antioxidative additive agent is ascorbic acid or a salt thereof and is administered in a dose of at least one of:
100 to 2000 mg of ascorbic acid per dosage unit,
600 to 1000 mg of ascorbic acid per dosage unit,
800 to 1000 mg of ascorbic acid per dosage unit, and
1000 mg of ascorbic acid per dosage unit.

8. The method according to claim 1, wherein the at least one antioxidative additive agent is glutathione or a salt thereof and is administered in a dose of at least one of:
100 to 1500 mg of glutathione per dosage unit,
100 to 1000 mg of glutathione per dosage unit,
500 to 800 mg of glutathione per dosage unit, and
600 mg of glutathione per dosage unit.

9. The method according to claim 1, wherein the at least one antioxidative additive agent is α-lipoic acid or a salt thereof, and is administered in a dose of at least one of:
100 to 1500 mg of α-lipoic acid per dosage unit,
100 to 1000 mg of α-lipoic acid per dosage unit,
500 to 800 mg of α-lipoic acid per dosage unit, and
600 mg of α-lipoic acid per dosage unit.

10. The method according to claim 1, wherein the at least one antioxidative additive agent is a selenium salt administered in a dose of at least one of:
50 μg to 500 μg of selenium per dosage unit,
100 μg to 300 μg of selenium per dosage unit,
200 μg to 300 μg of selenium per dosage unit, and
300 μg of selenium per dosage unit.

11. The method according to claim 1, wherein the at least one antioxidative additive agent is a zinc salt administered in a dose of at least one of:
1 mg to 100 mg of zinc per dosage unit,
5 mg to 50 mg of zinc per dosage unit, and
10 mg to 25 mg of zinc per dosage unit.

12. The method according to claim 1, wherein the at least one antioxidative additive agent is folic acid or a derivative or a salt thereof, and is administered in a dose of at least one of:
100 μg to 10 mg of folic acid per dosage unit,
1 mg to 10 mg of folic acid per dosage unit,
3 mg to 6 mg of folic acid per dosage unit, and
5 mg of folic acid per dosage unit.

13. The method according to claim 1, wherein the at least one antioxidative additive agent is vitamin B12 or a derivative thereof, and is administered in a dose of at least one of:
100 to 1500 mg of vitamin B12 per dosage unit,
100 to 1000 mg of vitamin B12 per dosage unit,
500 to 1000 mg of vitamin B12 per dosage unit, and
1000 mg of vitamin B12 per dosage unit.

14. The method according to claim 1, wherein the at least one antioxidative additive agent is a magnesium salt administered in a dose of at least one of:
100 to 1000 mg of magnesium per dosage unit,
100 mg to 500 mg of magnesium per dosage unit, and
400 mg to 500 mg of magnesium per dosage unit.

15. The method according to claim 1, wherein the at least one antioxidative additive agent is a potassium salt administered in a dose of at least one of:
100 mg to 1000 mg of potassium per dosage unit,
500 mg to 1000 mg of potassium per dosage unit,
600 to 800 mg of potassium per dosage unit, and
780 mg of potassium per dosage unit.

16. The method according to claim 1, wherein the at least one antioxidative additive agent is L-arginine or a salt thereof, and is administered in a dose of at least one of:
100 mg to 2000 mg of L-arginine per dosage unit,
300 mg to 1000 mg of L-arginine per dosage unit,
300 to 600 mg of L-arginine per dosage unit, and
450 mg of L-arginine per dosage unit.

17. The method according to claim 1, wherein the at least one antioxidative additive agent is administered at staggered times and is at least one of:
selenium salt in a dose of at least one of:
50 μg to 500 μg of selenium per dosage unit,
100 μg to 300 μg of selenium per dosage unit,
200 μg to 300 μg of selenium per dosage unit, and
300 μg of selenium per dosage unit, and
zinc salt in a dose of at least one of:
1 mg to 100 mg of zinc per dosage unit,
5 mg to 50 mg of zinc per dosage unit, and
10 mg to 25 mg of zinc per dosage unit.

18. The method according to claim 17, wherein the at least one of the at least one antioxidative additive agent is administered orally.

19. The method according to claim 17, wherein the at least one antioxidative additive agent is administered daily over a period of at least one of:
about 8 weeks after an infusion interval,
about 4 weeks after the infusion interval, and
about 3 weeks after the infusion interval.

20. The method according to claim 1, wherein the at least one antioxidative additive agent is ascorbic acid and glutathione.

21. The method according to claim 1, wherein the at least one antioxidative additive agent is ascorbic acid and magnesium.

22. The method according to claim 21, wherein the ascorbic acid and magnesium are administered simultaneously,
the ascorbic acid is administered in a dose of at least one of:
100 to 2000 mg of ascorbic acid per dosage unit,
600 to 1000 mg of ascorbic acid per dosage unit,
800 to 1000 mg of ascorbic acid per dosage unit, and
1000 mg of ascorbic acid per dosage unit, and
the magnesium is administered in a dose of at least one of:
100 mg to 1000 mg of magnesium per dosage unit,
100 mg to 500 mg of magnesium per dosage unit, and
400 mg to 500 mg of magnesium per dosage unit.

23. The method according to claim 1, wherein the at least one antioxidative additive agent is ascorbic acid, glutathione, α-lipoic acid, and vitamin B12.
